(19) United States
(12) Patent Application Publication
(10) Pub. No.: US 2003/0176795 A1
(43) Pub. Date: Sep. 18, 2003

(54) Blood pressure measurement apparatus and method

(76) Inventors: Thomas John Harris, Surrey (GB); Philip Needham, Berkshire (GB)

Correspondence Address: SCHWEGMAN, LUNDBERG, WOESSNER & KLUTH, P.A., P.O. BOX 2938, MINNEAPOLIS, MN, 55402, US

(21) Appl. No.: 10/307,804

(22) Filed: Dec. 2, 2002

Related U.S. Application Data
(63) Continuation of application No. PCT/GB01/02114, filed on May 15, 2001.

(30) Foreign Application Priority Data
Dec. 13, 2001 (WO) 01/93758 A1
Jun. 2, 2000 (GB) 0013504.6

(51) Int. Cl. A61B 5/02

(52) U.S. Cl. 600/485

(57) ABSTRACT

A blood pressure measurement apparatus and method is disclosed in which the taking of blood pressure measurements of a patient is controlled in dependence upon the analysis of a signal from the patient indicative of cardiac contractions.

The present invention generally relates to a blood pressure measurement apparatus and method and more particularly to a blood pressure measurement apparatus and method in which the taking of a blood pressure measurement of a patient is controlled on the basis of the analysis of a signal from the patient indicative of contractions of the heart of the patient.

Blood pressure describes the internal pressure within the arterial system of the body and is of genera interest as a significant prognostic and diagnostic indicator. Blood pressure is described in two values, the systolic and diastolic values. These two values describe the peak and trough values within a pulse cycle. The cycle is caused by the cyclic pumping action of the heart The peak value in the cycle corresponds to the pressure achieved just after the ventricles have contracted (systole) and the trough value represents the underlying or residual pressure in the system between systole.

Blood pressure is traditionally measured at the blachial artery in the upper arm Blood pressure measurements are conventionally manually taken using an inflatable sphygmomanometer cuff placed over the brachial artery on the upper arm. The cuff is inflated until the radial pulse at the wrist is no longer palpable. In this event the pressure in the cuff exceeds the arterial blood pressure. The diaphragm of a stethoscope is placed on the brachial artery just below the cuff. The cuff pressure is gradually reduced until sounds (Korotkoff sounds) can be heard. These sounds are caused by blood passing through the compressed artery at the peak of the arterial pressure cycle. At this point the arterial pressure is just greater an the cuff pressure and can be read off the pressure indicator. This is the systolic press. The cuff is then further deflated until the sounds first become muffled then disappear. At this point, blood flow is continuous in the artery as the lower pressure in the arterial pressure cycle is now just greater than the cuff pressure and can be read off the pressure indicator. This is the diastolic pressure.

There are three basic technologies for automated measurements. All of these can be applied to arm cuffs. Wrist and even finger Gut have been marketed but wit limited accuracy because pressure measurements made at the wrist or finger must be calibrated as brachial pressure equivalents.

In one prior art automatic blood pressure measurement technique, termed oscillometry, a cuff is inflated and the pressure released gradually. A pressure transducer records the curve of pressure release and analysis and algorithms detect points in the curve where the arterial pressure is evident in the deflation curve, Although is approach is low cost, it requires the patient to be completely still while the measurement is taken. Any arm movement or even muscle flexing will affect the pressure curve and invalidate the result. Thus this technology is not ideally ted to ambulatory monitoring.

A second prior art technique for automatic blood pressure measurement is the automation of the manual method and is termed auscultatory. In this method the cuff is inflated and the pressure reduced gradually. A microphone in the cuff listens for the Korotkoff sounds over the brachial artery and 11 pressure is read by a transducer. This method is more reliable than the oscillometry technique and is more forgiving to muscle and body movement This technique is used in ambulatory devices but is, however, more expensive and complex to fit as the microphone position is important.

A third prior art technique uses R wave gating of the auscultatory measurements. R wave gating uses conventional ECG electrodes to isolate the R wave and his is used to turn on the microphone for 150 msec. This helps reduce noise which can be mistaken for Korotkoff sounds by only listening to the sounds in the time period when they are genuinely produced. This method is even more reliable and provides the capability to measure blood pressure in active patients. It is however more expensive and more complex to fit as the patient has to wear 3 ECG electrodes on their chest in addition to the cuff.

An extension of the R wave gating technique is termed dynamic 3D K sound analysis in which the data is collected and arranged as a waterfall plot to detect when the Korotkoff sounds start and stop and reject the noise.

U.S. Pat. No. 5,322,069 discloses an automatic blood pressure measuring arrangement in which an analysis of the functioning of the heart is used to trigger blood pressure measurements. In particular, beat timings determined from an ECG signal are used to identify arrhythmias. When a predetermined arrhythmia is detected, the blood pressure measuring apparatus is controlled to take a blood pressure measurement.

It is known in the prior art that the ECG signal of a patient can provide information on the condition of the patient's heart. It is further known that the ECG signal can be automatically analysed to provide information on the functioning of the heart. For example, in European patent no. 850016, the contents of which are hereby incorporated by reference, it is disclosed that an ECG signal can be automatically analysed by extracting features of the ECG signal and using the features as input feature vectors to a neural network. To the inventors' knowledge, however, none of this technology has however been applied to blood pressure measurements.

All of these prior art blood pressure measuring techniques suffer from the limitation that no account is taken of the functioning of the heart when the blood pressure measurements are taken.

JP-A-9220206 discloses a blood pressure measurement system in which a blood pressure measurement is stopped if the heart rate changes by more than a predetermined amount during the blood pressure measurement. Although this system takes some amount of heart functioning, it only does this after starting a blood pressure measurement. There is no consideration given as to whether it is a suitable time to take a blood pressure measurement. This system can thus result in multiple abortive attempts to take a blood pressure measurement which can be distressing to the patient particularly since the taking of a blood pressure measurement usually involves inflating a sphygmomanometer cuff placed over the upper arm of the patient at some discomfort.

The present invention thus provides a blood pressure measurement apparatus and method in which the taking of blood pressure measurements of a patient is controlled by the analysis of a signal from the patient indicative of cardiac contractions.

The present inventors have realised that information on the functioning of the heart available from cardiac measurements can provide an enhanced blood pressure measuring technique.

The signal which is indicative of cardiac contractions can be obtained from the patient using many different known techniques. For example, acoustic signals can be obtained and analysed in order to indicate the functioning of the heart. However, the most convenient technique for monitoring the functioning of the heart is electrophysiologically. For example, a 2 electrode strap be placed around the chest of the patient to provide electrocardiac measurements enabling the detection of R waves. These simple electrocardiac measurements can be used to measure pulse rate and pulse intervals. The pulse rate and pulse interval information can then be used to control the taking of blood pressure measurements.

Another common technique for determining the functioning of the heart of the patient is the full electrocardiac detection technique which generates an electrocardiograph signal. As is known in the prior art example in European patent no. 850016, the electrocardiograph signal contains cardiac function information on not only cardiac rhythm but also cardiac conditions. Thus, the ECG signal can be used to determine not only rhythm information but also information on cardiac conduction controlling cardiac muscle activity. ECG signal detection apparatus is however more complex than the simple electrocardiac strap used to determine cardiac pulse rate and cardiac pulse intervals by simple R wave detection.

Thus the inventors have realised that any conventional technique for obtaining information on the functioning of the heart can be used to control when blood pressure measurements are taken.

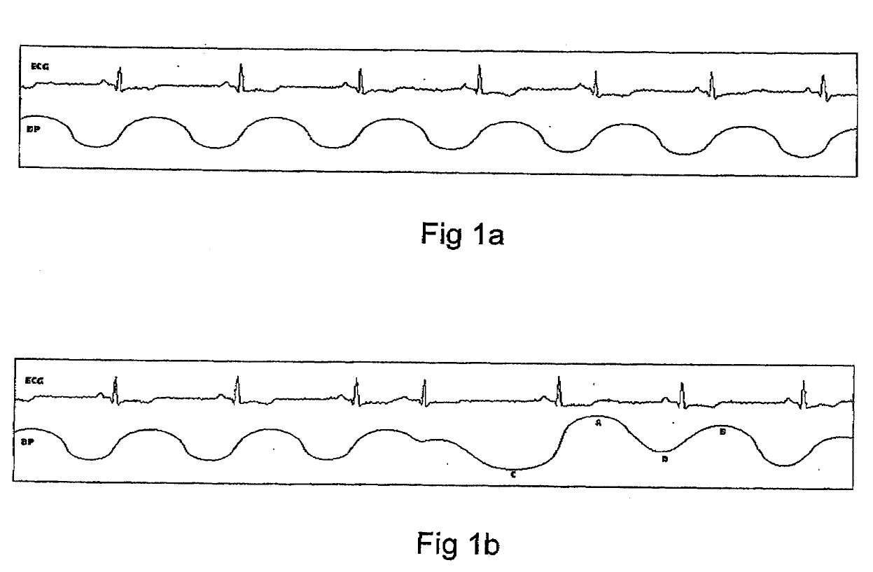

For example, the inventors have realised that there are variations in the arterial pressure cycle caused by cardiac arrhythmia. FIG. 1a illustrates the pressure curve and ECG trace during a consistent rhythm. During this rhythm the pressure cycle is unchanged from beat to beat and any of the peaks or trough measured would give an accurate indication of the true blood pressure. However, where the cardiac cycle is arrhythmic, e.g. there is a single premature beat, as can be seen in FIG. 1b, the single premature beat at the centre of the ECG trace has caused an additional pressure peak. However, of greater significance is the effects during the recovery stage. Point A shows the increased pressure caused by the next heartbeat. This is increased from normal because more blood has been ejected from the heart. This increase in pressure takes a few beats to correct as can be seen from point B. Point C shows that in the elongated pause between the premature beat and the next, the diastolic pressure is lower than normal. During the recovery stage, the diastolic pressure is also higher than normal, as can be seen at point D.

Thus blood pressure readings take during This process would give an inaccurate reflection of the underlying blood pressure. Cardiac rhythms and conditions during which accurate blood pressure measurements can be taken comprise, for example, sinus rhythm, sinus tachycardia, sinus bradycardia, pre-excitation beats, low heart rate variability, reentrant tachycardia, supra ventricular tachycardia (SVT), and junctional rhythm. Cardiac rhythms and conditions which would lead to curate blood pressure measurements include, for example, premature atrial contractions, premature ventricular contractions, a dropped beat, aberrantly conducted beat, sinus pause, sinus arrest, atrial bigeminy or trigeminy, ventricular bigeminy or trigeminy, atrial fibrillation, and flutter.

Thus, in an aspect of the present invention, the analysis of the received signal is used to confirm that the current cardiac cycle is suitable for an accurate blood pressure measurement to be taken. There is thus an external trigger fox a blood pressure measurement Such an external trigger can comprise a periodically generated signal, e.g. a timed signal, or a manually generated signal, such as when a patient requests the tan of a blood pressure measurement when they feel unwell. Before a blood pressure measurement is taken in response to a request signal, the received signal is analysed to identify whether it is a suitable time for blood pressure measurement to be taken. A blood pressure measurement is only taken if the analysed signal indicates that it is suitable to do so. The analysis can take the form of identifying cardiac conditions and/or cardiac rhythms as a classification of the cardiac conditions and/or cardiac rhythms as suitable or unsuitable for a blood pressure measurement.

In a preferred embodiment of the present invention, the analysis is carried out using a number of neural networks. Each neural network represents a cardiac condition classification which can be determined from the morphology of the ECG signal for a cycle and each cardiac cycle can be classified accordingly. Also, cardiac rhythm information can be extracted from the received signal and used together with the cardiac condition classification obtained from the neural networks to identify the cardiac conditions and/or cardiac rhythms.

In addition to determining whether or not to take a blood pressure measurement using the analysis of the received signal, preferably the analysis is continued during a blood pressure measurement and the continued analysis is used to determine whether the blood pressure measurement should continue or be aborted. Since the taking of a blood pressure measurement will span several cardiac cycles, it is desirable to identify when an unsuitable cardiac cycle occurs during a blood pressure measurement so that an inaccurate blood pressure measurement can be avoided by aborting the measurement Alteratively, if the cardiac event which occurs during the blood p a measurement is not too severe and the normal cardiac rhythm will recover quickly, as an alternative to aborting the blood pressure measurement, the measurement can instead be suspended to await the next suitable cardiac cycle.

The cardiac signal can be received and analysed continuously. Alternatively, the analysis of the received signal only takes place when the request signal for a blood pressure measurement is generated and preferably the analysis continues whilst the blood pressure measurement is being taken. This triggering of the analysis -of the cardiac signal is particularly suited to a portable apparatus which has limited battery power. By only processing data when necessary, power is conserved.

In preferred embodiments of the present invention, the blood pressure measurements taken can be stored. The cardiac signal ear also be stored continuously, or only the cardiac signals associated with blood pressure measurements be stored. Also, the results of the analysis of the cardiac signal can also be stored.

An embodiment of the present invention can also use the analysis of the received signal to identify predetermined cardiac events for which a blood pressure measurement is desired and a blood pressure measurement is taken when a predetermined cardiac event is identified.

Thus in this embodiment of the present invention, a trigger for the taking of a blood pressure measurement is the identification of a particular cardiac event. This allows blood pressure measurements to be taken that coincide with cardiac rhythms or conditions of interest Common cardiac rhythm and conditions for which the coincident blood pressure measurement will be s for diagnostic, therapeutic and prognostic reasons include, for example, sinus tachycardia, sinus bradycardia, paroxysmal atrial fibrillation, heart block myocardial ischaemia, paroxysmal supraventricular tachycardia.

This embodiment of the present invention requires the real time processing of the cardiac signal for the fill duration of a test.

This embodiment of the present invention cm be used in conjunction with the first aspect of the preset invention to provide a blood pressure measurement arrangement in which periodic blood pressure measurements are taken so long as the cardiac cycle is suitable, and in addition, where predetermined cardiac events occur which are of interest, additional blood pressure measurement can be taken.

The present invention is applicable to both oscillometric and auscultatory blood pressure measurements.

The preset invention is also applicable to the use of R wave gating in an auscultatory blood pressure measuring system.

The present invention is applicable to any form of blood pressure measuring arrangement, e.g. both static and ambulatory. The present invention is, however, particularly suited to ambulatory blood pressure measurement when used in conjunction with an auscultatory blood pressure measuring system.

Embodiments of the present invention will now be described with reference to the accompanying draw , in which:

FIG. 1a is a graph of the normal cyclic behaviour of the blood pressure curve with a normal ECG;

FIG. 1b is a graph showing the effect on the arterial pressure cycle of a single premature beat;

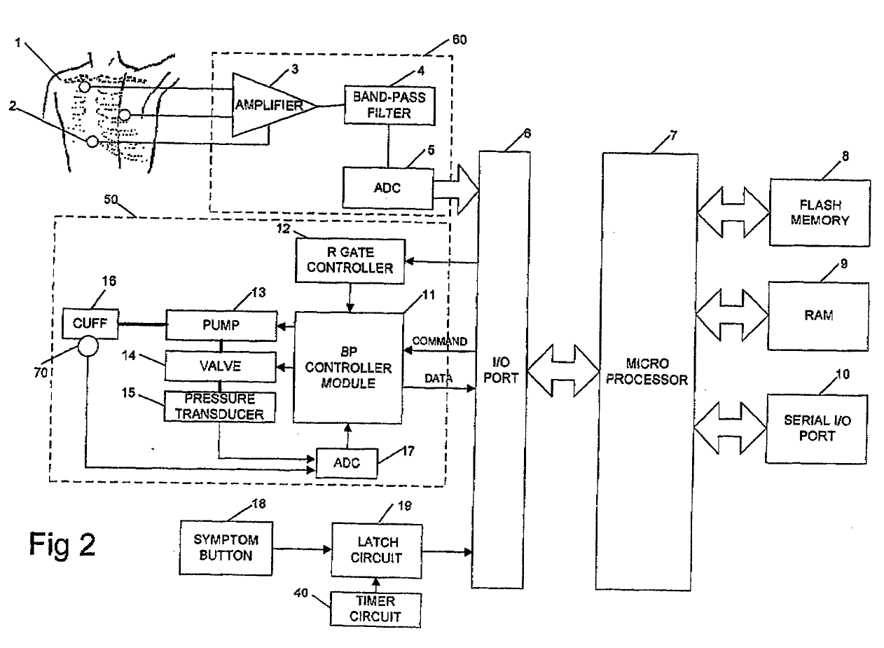

FIG. 2 is a schematic diagram of a first embodiment of the present invention;

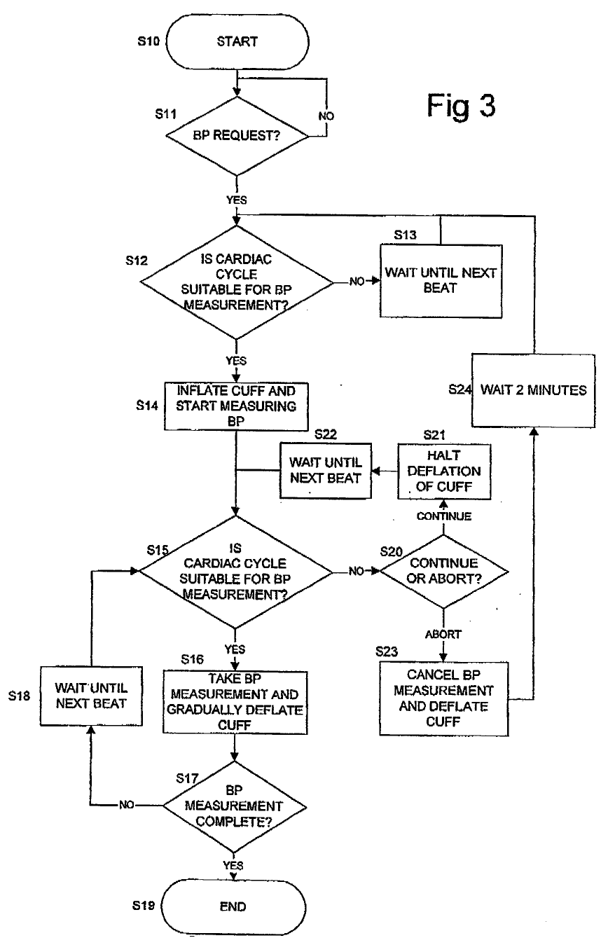

FIG. 3 is a flow diagram illustrating the implementation of the first embodiment of the present invention;

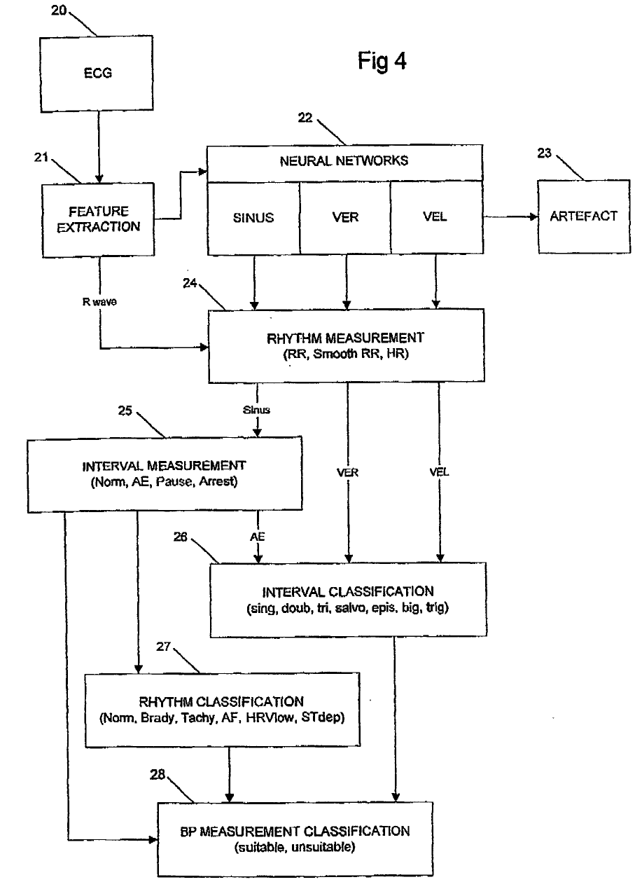

FIG. 4 is a functional diagram illustrating the analysis and classification of the ECG signal in the first embodiment of the present invention;

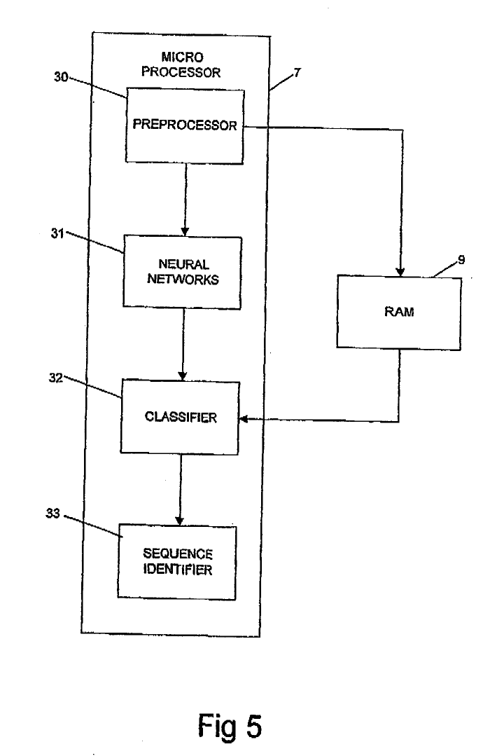

FIG. 5 is a functional block diagram of the processes implemented by the microprocessor in accordance with the first embodiment of the present invention;

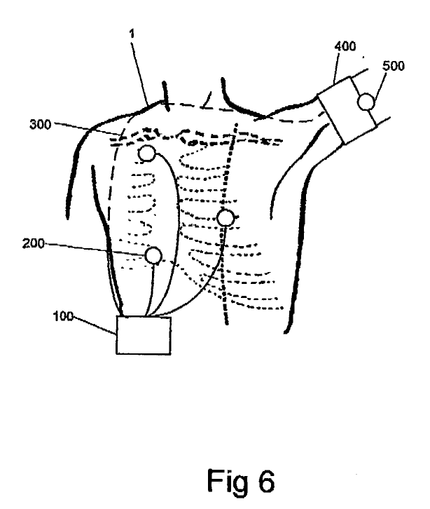

FIG. 6 is a diagram illustrating an ambulatory blood pressure monitoring device in accordance with the first embodiment of the present invention;

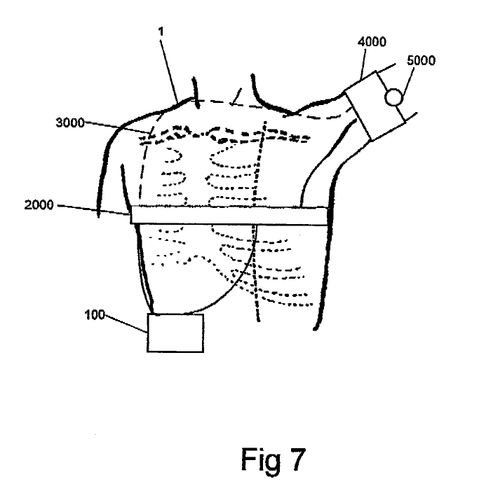

FIG. 7 is a diagram illustrating an ambulatory blood pressure monitoring device in accordance with a second embodiment of the present invention;

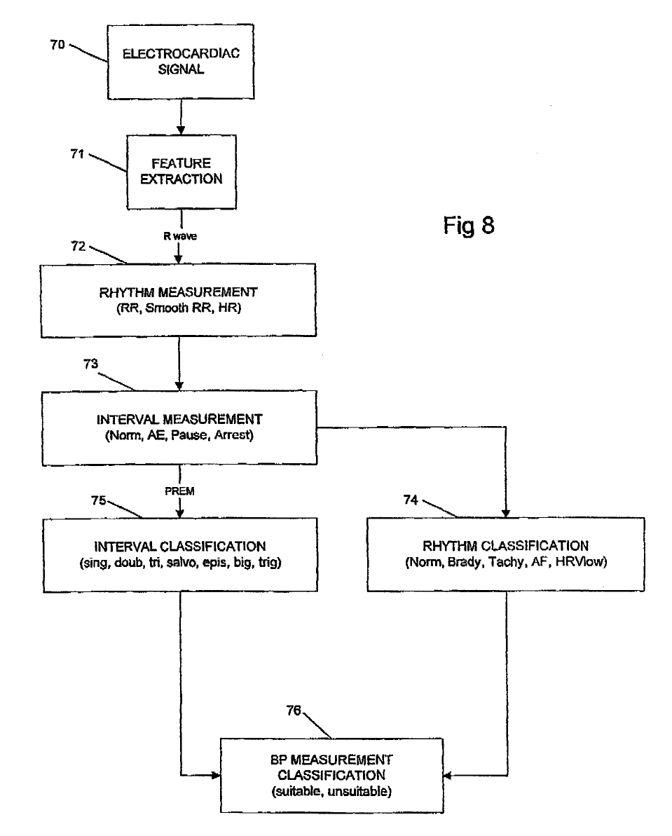

FIG. 8 is a functional diagram illustrating the analysis and classification of the electrocardiac signal in the second embodiment of the present invention; and

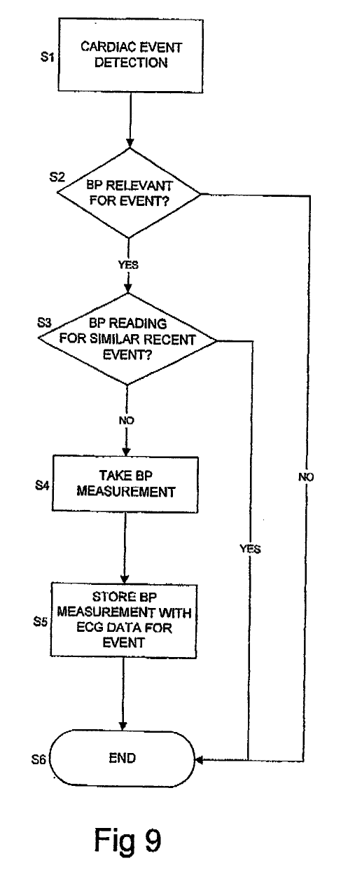

FIG. 9 is a flow diagram illustrating a method of operation of a blood pressure monitoring apparatus in accordance with a third embodiment of the present invention.

A first embodiment of the present invention will now be described with reference to FIGS. 2 to 6.

FIG. 2 is a schematic diagram of a blood pressure measurement device in accordance with a fast embodiment of the present invention As can be seen from FIG. 2, the device incorporates a blood pressure measurement unit 50 and an ECG measurement unit 60.

The patient 1 has electrodes 2 arranged on their chest to obtain an ECG signal. The ECG signal is input to an amplifier 3 and the output of the amplifier 3 is input to a bandpass filter for noise reduction. The bandpass filtered signal is input to analogue-to-digital converter 5 for input into the input/output port 6 of the device. In this way a digitized input/output signal is received by the device.

A microprocessor 7 is provided for implementing the analysis of tie electrocardiograph signal. The microprocessor is provided with flash memory 8 containing a program code for controlling the operation of the microprocessor. Random access memory (RAM) 9 is also provided for use as working memory by the microprocessor 7. The device is also provided with a serial input/output port 10 for communicating with a remote device such as a printer or a host computer. ECG data, blood pressure measurements, and analysed ECG data can be stored in the flash memory 8. The recordal of the ECG and the analysis of the FCC is preferably implemented in the same manner as disclosed in co-pending United Kingdom patent application no. 0011196.3 entitled “Hear Monitoring Apparatus and Method”. In such an arrangement, the RAM 9 is used as a cyclical memory for the low power storage of the ECG signal on a temporary basis. The device includes a symptom button 18 which sets a latch in a latch circuit 19. Tis enables the input of a manual request for blood pressure measurement Alternatively, or in addition, a timer circuit 40 can be provided for setting the latch in the latch circuit 19. The latch is read via tee input/output port 6 by the microprocessor 7 and when the latch is set, the microprocessor can initiate processing of the electrocardiograph signal stored in the RAM 9. The electrocardiograph signal can then be transferred to the flash memory 8 for non-volatile storage together with the analysed electrocardiograph signal. If the analysis of the electrocardiograph signal by the processor 9 indicates that a blood pressure measurement Should be taken, a command is output to a blood pressure controller module 11. Also, as a result of the analysis of the electrocardiograph signal, the R waves of the electrocardiograph signal can be identified and ting signals identifying the R waves can be output to an R gate controller 12. The R gate controller 12 generates R gate signals for input to the blood pressure controller module 11. The blood pressure controller module 11 controls a pump 13, a valve 14 and a pressure transducer 15, all of which are connected to a cuff 16. To initiate a blood pressure measurement, the blood pressure controller module 11 causes the pump 13 to pump up the cuff 16. When a predetermined pressure is reached, the pump stops. The pressure transducer 15 outputs a pressure signal to an analogue-to-digital converter 17 which inputs a digitized value to the blood pressure controller module 11. The cuff 16 is also provided with a microphone 70 for detecting Korotkoff sounds. These are input to the analogue-to-digital converter 17. The valve 14 will be controlled to gradually reduce the pressure in the cuff and this pressure is measured together with the output of the microphone 70.

The result of tee analysis within the blood pressure controller module 11 will be a blood pressure read comprising a systolic and diastolic pressure.

The command to the blood pressure controller module 11 can comprise not simply a command to start a blood pressure reading, but also a command to suspend the blood pressure measurement when an unsuitable cardiac cycle occurs. In such a case, the blood pressure controller module 11 controls the valve 14 to suspend the deflation of the cuff 16. Additionally, the pump 13 may be activated to slightly re-inflate the cuff 16.

The blood pressure measurement device of this embodiment of the present invention comprises an ambulatory blood pressure measurement device 100 which can be worn by the patient 1 as illustrated in FIG. 6. The device is carried, for example clipped to a belt, with a pipe 300 leading around the back of the patient 1 and across the shoulders to the cuff 400 provided on the arm of the patient 1. ECG electrodes 200 are placed on We chest of the patient 1 and connected to the device 100 A microphone 500 is provided with the cuff 400 to detect the Korotkoff sounds. The detected sounds are transmitted by a wire carried with the pipe 300 to the device 100 for analysis.

It can thus be seen that the device comprises a compact monitoring device which can be worn for a length of time by a patient to enable blood pressure measurements to be taken over a long test period.

The operation of this embodiment of the present invention will now be described with reference to the flow diagram of FIG. 3.

In step S10 the device is initiated and in step S11 the device awaits a blood pressure request signal. This can be provided either from a patient's manual request or from timed signals. When a blood pressure request signal is received, in step S10, it is determined whether the cardiac cycle is suitable for blood pressure measurement For example, cardiac conditions and rhythms which are unsuitable for blood pressure measurements include premature atrial contractions, premature ventricular contractions, a dropped beat, aberrantly conducted beat sin pause, sinus arrest, atrial bigeminy or trigeminy, ventricular bigeminy or trigeminy; atrial fibrillation and flutter, Cardiac rhythms and conditions which are suitable for blood pressure measurements include sinus rhythm, sinus tachycardia, sinus bradycardia, pre-excitation beats, low heart rate variability, reentrant tachycardia, supra ventricular tachycardia (SVT), and junctional rhythm. A more detailed discussion on how the ECG signal is analysed to identify suitable and unsuitable cardiac events will be described hereinafter in more detail.

Thus, if in step S12 it is determined that the cardiac cycle is unsuitable for blood pressure measurements, in step S13 the process waits Until the next cardiac cycle before once again returning to step S12 to determine whether the cardiac cycle is suitable for blood pressure measurement.

When a suitable cardiac cycle is eventually identified, in step S14 the cuff worn by the patient is inflated and the blood pressure measurement is started. In step S15 it is continuously determined whether the cardiac cycle is suitable for blood pressure measurement. Whilst it is, in step S16 the blood pressure measurement is taken and the cuff is continuously and gradually deflated. In step S17 it is determined whether the blood pressure measurement is complete and if so the process halts in step S19. If not, in step S18 the next cardiac cycle is awaited for the process in step S15 to once again determine whether the cardiac cycle is suitable for blood pressure measurements.

If at any time during the blood pressure measurement it is determined that the cardiac cycle is unsuitable, in step S20 it is determined whether the cardiac cycle requires that the blood pressure measurement should be aborted or whether it is still possible to continue with the blood pressure measurement. For example, if the analysis of the electrocardiograph signal determined that there had been atrial fibrillation, since it would take a considerable amount of time for the cardiac cycle to return to a rhythmic cycle, the blood pressure measurement is cancelled in step S23 and the cuff is deflated. In step S24 the process then waits 2 minutes to allow for arterial recovery before returning to step S12 to once again try and restart a blood pressure measurement if however in step S20 the cardiac event which occurred was a single ectopic beat, it would be decided that the blood pressure measurement could be continued and thus in step S21 the deflation of the cuff would be halted (and the cuff could be slightly reinflated) and in step S22 the process would wait until the next cardiac cycle before returning to step S15 to dee whether the next cardiac cycle is suitable for blood pressure measurement Thus this process has a suspensory effect on the blood pressure measurement and allows the process to continue whilst ignoring unsuitable beats which will not have a lasting effect on the cardiac rhythm.

It can thus be seen from the flow diagram of FIG. 3 that although an external blood pressure request is generated, a blood pressure measurement is not taken whilst the cardiac function could cause an inaccurate blood pressure measurement.

The process by which the electrocardiograph signal is analysed in order to determine whether the cardiac function is suitable or unsuitable for blood pressure measurement will now be described with reference to FIG. 4.

FIG. 4 illustrates the functions performed by the microprocessor 7 in determining whether the cardiac function is suitable or unsuitable for a blood pressure measurement. The electrocardiograph input function 20 receives the input digitized electrocardiograph signal. The electrocardiograph signal then undergoes feature extraction 21 to extract the salient features of the electrocardiograph signal. These features can comprise measurements of the height and spacing between peaks or features of a cycle of the electrocardiograph signal. A suitable feature extraction technique is disclosed for example in European patent no. 850016. The extracted features which comprise values defining heights and separations are then used as a feature vector for input into three neural networks 22. One neural network is provided in which reference feature vectors identify sinus beats, i.e. normal shaped beats with pqrst waves. A second neural network is provided to identify a ventricular ectopic b with a shape thought to be characteristic of a right ventricular origin (VER). The third neural network is provided to identify a ventricular etopic beat with a shape thought to be characteristic of a left ventricular origin (VEL). If any input ECG cycle having undergone feature extraction cannot be classified by one of the three neural networks, it can be output into a classification defining it as an artefact 23, Thus, the neural networks 22 enable artefact detection by the detection and rejection of noise spikes.

A rhythm measurement function 24 receives the resultant classification of the ECG cycle as well as R wave values from the feature extraction function 21. Thus the rhythm measurement function is able to determine the time interval between two successive R waves (RR), a mean or median of the i few RR intervals (smooth RR) and the heart rate (HR). The rhythm measurement function 24 generates these measurements for all classifications of the input ECG cycle. If the input ECG cycle is classified as a sinus beat, the rhythm measurement function 24 outputs the measurements to an interval measurement function 25 which provides interval measurements and identifies whether the interval is within a normal band for the current heart rate (Norm), there is an atrial ectopic beat or premature beat, i.e. a beat rhythm RR of 0.7 smooth RR, the RR is 1.7 seconds or more (pause), or the RR interval is 3 seconds or more (arrest). If an atrial ectopic beat (AE) or premature is identified, this is indicated to an interval classification function 26. The interval classification function 26 also receives the output of the rhythm measurement function 24 for VER classifications or VEL classifications. The interval classification function classies beats as a single isolated event (sing), a double of two events in a row (doub), a triple comprising three events in a row (tri), between four and seven events in a row (salvo), an episode comprising a run of at least eight events in a row (epis), bigeminy comprising alternating events and normals in a series (big), or trigeminy comprising events separated by two normals in a series (trig).

If the interval measurement function 25 identifies normal rhythm, the rhythm classification function 27 classifies the beat into normal, bradycardia (slower than 50 beats per minute), tachycardia (faster than 120 per minute), atrial fibrillation (AF), a low variability in successive RR intervals such that the heart does not adjust to breathing (HRVlow) and ST segment depression in which the region between the S wave and the T wave is depressed below the isoelectric line, which is indicative of ischaemia (STdep).

The results of the rhythm classification function 27, the interval measurement function 25 and the interval classification function 26 are input to a blood pressure measurement classification function 28 to determine whether the cardiac cycle is suitable or unsuitable for a blood pressure measurement. The blood pressure measurement classification function 28 call determine that the cardiac cycle is unsuitable for blood pressure measurements when the cardiac cycle is classified as: pause, arrest AF, AE or VE as single, double, triple, salvo, big or trig. The blood pressure measurement classification function 28 can determine that the cardiac cycle is suitable for blood pressure measurement when the cardiac cycle is classified as: normal, tachy, brady, HRVlow, episodic, AE or VE, or STdep.

Thus, functions 21 to 28 illustrated in FIG. 4 comprise analysing means in this embodiment of the present invention in order to analyse the electrocardiograph signal in order to determine whether the cardiac cycle is suitable or unsuitable for a blood pressure measurement. This process is applicable to both steps S12 and S15 in FIG. 3 and can thus be used not only to determine whether to start taking a blood pressure measurement but also whether to continue with the blood pressure measurement.

FIG. 5 illustrates the functions performed within the microprocessor 7 by the program code read from the flash memory 8. A preprocessor module 30 carries out the feature extraction process and stores the extracted features in RAM 9. Also, the extracted features are input to neural networks 31 implemented by a program code in the microprocessor 7. A classifier 32 is also implemented by a program code in the microprocessor 7 to carry out the functions 24 to 28 of FIG. 4. A sequence identifier 33 is also provided to store the classifications output from the classifier 32 over a plurality of previous cardiac cycles. The sequence identifier comprises the final step in the decision of whether the cardiac cycle is suitable for blood pressure measurement (steps S12 and S15 in FIG. 3). It is not sufficient that the current cycle is suitable for blood pressure measurement. It is also necessary to look back over a few previous cardiac cycles to ensure that the effects of any previous cardiac irregularity has decayed sufficiently for the cardiac cycle to return to a stable rhythmic pattern.

The second embodiment of the present invention will now be described with reference to FIGS. 7 and 8.

In this embodiment of the present invention, instead of using a full electrocardiograph signal, an electrocardiac signal is obtained from a chest strap 2000 containing two electrodes and worn on the chest of the patient 1 as can be seen in FIG. 7. The electrocardiac signal input to a device 1000 carried by the patient 1. The electrocardiac measurements obtained from the chest strap 2000 are used to determine heart rate and pulse intervals from the electrical activity of the heart by identifying R waves, This information is used to control when a blood pressure measurement is taken in a similar manner to the process illustrated to the flow diagram of FIG. 3 for the first embodiment of the present invention A cuff 4000 is won by the patient 1 and carries a microphone 5000. The microphone and cuff communicate with the device 1000 via by a pipe and wire 3000. Thus the second embodiment of the present invention is similar to the first embodiment except that because a chest step 2000 is used in place of the ECG electrodes, a simpler arrangement is provided for.

Since an electrocardiac signal is provided which does not provide a full electrocardiograph signal, the decision on whether or not the cardiac cycle is suitable for blood pressure measurement is taken based on the electrocardiac signal which can only provide an indication of cardiac rhythm and not cardiac morphology.

The apparatus of the second embodiment of the present invention is thus similar to the apparatus of the fist embodiment of the preset invention except that the separate multiple ECG electrodes are replaced with a chest strap containing two electrodes.

Thus in accordance with the second embodiment of the present invention, as with the first embodiment of the present invention, although an external blood pressure request is generated, a blood pressure measurement is not taken whilst the cardiac function would cause an inaccurate blood pressure measurement.

The process by which the electrocardiac signal is analysed in order to determine whether the cardiac function is suitable or unsuitable for blood pressure measurement will now be described with reference to FIG. 8.

FIG. 8 illustrates the functions performed by the microprocessor 7 in determining whether the cardiac function is suitable or unsuitable for blood pressure measurement in accordance with the second embodiment of the present invention. The electrocardiac signal input function 70 receives the input degitised electrocardiac signal. The electrocardiac signal then undergoes feature extraction 71 to extract the R waves to identify the timing of the contraction of the heart. The R waves detections are then input into the rhythm measurement function 72 in order to determine the timing between two successive R waves (RR), in mean or median of the last few RR intervals (smooth RR) and the heart rate ant). The rhythm measurement function 72 outputs the rhythm measurements to an interval measurement function 73 which identifies whether the interval between heart beats is within a normal band with the current heart rates (Norm), which is a premature beat ie a beat rhythm RR of 0.7 smooth RR; (em), the RR is 1.7 secs or more (pulse), or the RR interval is 3 secs or more) If a premature beat is identified, this is indicated to an interval classification function 75 which classifies beats as a single isolated event (sing), a double ie two events in a row (doub), a triple comprising three events in a row (tri), between our and seven events in a Tow (salvo), an episode comprising a run of at least eight events in a row (epis), bigeminy comprising alternating events and normals in a series (big), or trigeminy comprising events separated by two normals in a series (trig).

If the interval measurement function 73 identifies the interval as nodal, pause or an arrest, a rhythm classification function 74 then classifies the rhythm of the beat into normal, bradycardia (slower than 50 beats per minute), tachycardia (fester than 120 beats per minute)) atrial fibrillation (AF), or a low variability in successive RR intervals such that the heart does not adjust to breathing (HRVlow).

The results of the rhythm classification function 74 and the interval classification function 75 are input to a blood pressure classification function 76 to determine whether the cardiac cycle is suitable or unsuitable for a blood pressure measurement The blood pressure measurement classification function can determine that the cardiac cycle is unsuitable for blood pressure measurements when the cardiac cycle is classified as: pause, arrest, AF, and premature beats of simple, double, triple, solvo, bigeminy and trigeminy. The blood pressure measurement classification 76 can determine that the cardiac cycle is suitable for blood pressure measurement and the cardiac cycle is classified as: normal, tachy, brady, HRVlow or epis.

Thus the functions 71 to 76 illustrated in FIG. 8 comprise analysing means in this embodiment of the present invention in order to analyse the electrocardiac signal in order to determine whether the cardiac cycle is suitable or unsuitable for a blood pressure measurement This process is applicable to both steps S12 and S15 in the flow diagram FIGS. 3 which is applicable to this embodiment of the present invention and can thus be used not only to determine whether to start taking the blood pressure measurements but also whether to continue with the blood pressure measurements.

In this embodiment of the present invention, the frictions performed by the microprocessor are reduced compared to the first embodiment since there are no neural networks used& A simplified pre-processor module identifies the R waves and acts as a classifier which classifies the cardiac cycle as described with reference to FIG. 8, and a sequence identifier looks over a previous number of cardiac cycles to identify whether the cardiac cycles of the previous time period indicate that any previous cardiac irregularity has decayed sufficiently for the cardiac cycle to return to a stable rhythmic pattern This indicates that it is now a suitable time to take a blood pressure measurement.

A third embodiment of the present invention will now be described with- reference to the flow diagram of FIG. 9. In this embodiment of the present invention, the taking of a blood pressure measurement is triggered by a cardiac event. The cardiac cycle is continuously read and analysed in order to detect a predetermined cardiac event, A blood pressure measurement is then taken to coincide with the cardiac event This is useful for diagnostic, therapeutic and prognostic reasons.

Examples of events which can be used to trigger blood pressure monitoring are: sinus tachycardia, sinus bradycardia, paroxysmal atrial fibrillation, atrial flutter, heart block, myocardial, ischaemia, paroxysmal supraventricular tachycardia, and patient symptomatic. In this embodiment of the present invention, the classification system as illustrated in FIG. 4 or FIG. 8 from the first or second embodiments respectively can be used.

The flow diadem of FIG. 9 illustrates the process which takes place for every cardiac cycle. In step S2 it is determined whether a blood pressure reading is relevant for such an event If not, the process totes in step S6. If the taking of a blood pressure measurement is relevant, in step S3 it is determined whether there is a similar blood pressure reading for a recent event If so, there is no need to retake the blood pressure measurement and the process terminates in step S6. If there is no similar blood pressure reading in step S4 the blood pressure measurement is taken and in step S5 the blood pressure measurement is stored with the electrocardiograph data and the analysed electrocardiograph data for the event. The process then terminates in step S6.

The apparatus used for blood pressure measurements in accordance with the id embodiment of the present invention can comprise the same apparatus as for the first embodiment of the present invention as illustrated in FIG. 2 and can comprise an additional function.

Thus in accordance with this embodiment of the present invention, the take of a blood pressure measurement is triggered not only by an external trigger but also by a suitable cardiac event which is identified by the analysis of the electrocardiograph signal. A blood pressure measurement can be stored together with the electrocardiograph signal and the analysed electrocardiograph data in memory for review at a later date. Thus the combined embodiment enables blood pressure measurements to be taken periodically, either automatically or manually. Also, blood pressure measurements can be taken automatically upon detection of a predetermined class of cardiac event.

In any of the embodiments, the blood pressure measurements can be stored in memory. Also, the electrocardiograph signal during the blood pressure measurements can be stored together with the results of the analysis of the electrocardiograph signal. In this way a wealth of information can be made available to a healthcare professional.

Although in the embodiments the blood pressure measurement request signal is generated (either manually or automatically) in the portable apparatus, the present invention encompasses any method of obtaining the signal such as by receiving it from another apparatus. For example, the signal could be transmitted from apparatus operated by a healthcare professional to trigger a blood pressure measurement by the apparatus. The transmission can be via a wireless link to the portable apparatus worn by a patient.

The present invention is particularly suited to au ambulatory device which can be worn by a patient for a length of time. By periodically taking blood pressure readings and storing cardiac data and analysed cardiac data, the power requirements of the device is greatly reduced compared to a continuous real time monitoring device. Further, the memory requirement of the device is reduced. Thus the present invention is particularly suited for use with the heart monitoring apparatus and method described in co-pending United Kingdom Pant application no, 00111963 entitled “Heart Monitoring Apparatus and Method”. However, the present invention is not limited to use with such an ECG monitoring device and can be used in conjunction with any suitable cardiac analysis equipment.

It will be apparent to a skilled person in the an that modification lie within the spirit and scope of invention.

1. Blood pressure measurement apparatus for measuring the blood pressure of a patient, the apparatus comprising:
receiving means for receiving a signal from the patient indicative of contractions of the heart of the patient;
analysing means for analysing the received signal;
blood pressure measuring means for measuring the blood pressure of the patient;
determining means for determining whether to take a blood pressure measurement on the basis of the analysis of the received signal by ad analysing means; and
control means for controlling the blood pressure measuring means to perform a blood pressure measurement if a blood pressure measurement request signal has been received by said control means and in dependence upon the detention by said determining means.

2. Blood pressure measurement apparats according to claim 1 including generating means for generating said blood pressure measurement signal.

3. Blood pressure measurement apparatus according to claim 2, wherein said generating means is adapted to automatically periodically generate said request signal.

4. Blood pressure measurement apparatus according to claim 2 or claim 3, wherein said generating means is manually operable.

5. Blood pressure measurement apparatus according to any preceding clam wherein said analysing means is adapted to identify whether it is a suitable time for a blood pressure measurement to be taken and said determining means is adapted to determine to take a blood pressure measurement only if said analysing means has identified the time to be suitable.

6. Blood pressure measurement apparatus according to claim 5, wherein said analysing means is adapted to identify cardiac conditions and/or cardiac rhythms and to classify said cardiac conditions and/or cardiac rhythms as suitable or unsuitable for a blood pressure measurement.

7. Blood pressure measurement apparatus according to claim 6, wherein said analysing means includes neural network means comprising a neural network for each of a plurality of cardiac classifications to clad the received signal based on its morphology, means for determining cardiac rhythm information from said received signal, and means for identifying cardiac conditions and/or cardiac rhythms using the cardiac classifications and the cardiac rhythm information.

8. Blood pressure measurement apparatus according to any preceding claim wherein said analysing means is adapted to continue to analyse the received signal during a blood pressure measurement, and said deter means is further adapted to determine whether a blood pressure measurement should continue or be aborted on the basis of the analysis of said received signal by said analysing means.

9. Blood pressure measurement apparatus according to claim 8, wherein said analysing means is adapted to identify whether functioning of the heart is suitable for continuance of a blood pressure measurement, and said determining means is adapted to determine to continue to take a blood pressure measurement only if said analysing means has identified the functioning of the heart to be suitable.

10. Blood pressure measurement apparatus according to claim 9, wherein said analysing means is adapted to identify cardiac conditions and/or cardiac rhythms and to classify said cardiac conditions and/or cardiac rhythm as suitable or unstable for a blood pressure measurement.

11. Blood pressure measurement apparatus according to any one of claims 8 to 10, wherein said determining means is further adapted to determine whether to suspend a blood pressure measurement for a number of cardiac cycles on the basis of the analysis of said received signal by said analysing means.

12. Blood pressure measurement apparatus according to any prig claim wherein said analysing means is further adapted to analyse the received signal to identify predetermined cardiac events for which a blood pressure measurement is desired, and said determining means is adapted to determine that-a blood pressure measurement is to be taken when said analysing mean identifies a said predetermined cardiac event.

13. Blood pressure measurement apparatus according to any preceding claim, wherein said analysing means is adapted to continuously analysed the received signal.

14. Blood pressure measurement apparatus according to any one of claims 1 to 11, wherein said analysing means is adapted to only perform the analysis of the received signal after said request signal has been received by said control means and whilst the blood pressure measurement is being taken.

15. Blood pre measurement apparatus according to any preceding claim wherein said receiving means is adapted to eve an electro-physiological cardiac signal.

16. Blood pressure measurement apparatus according to any preceding claim wherein said analysing means is adapted to analyse the received signal to determine pulse rate and pulse intervals.

17. Blood pressure measurement apparats according to any preceding wherein said receiving means is adapted to re an ECG signal from the patient.

18. Blood pressure measurement apparatus according to any preceding claim, including storage means for sting blood pressure measurements.

19. Blood pressure meant apparatus according to any preceding claim, including signal storage means for storing the received signal.

20. Blood pressure measurement apparatus according to claim 19, wherein said signal storage means is adapted to only store the received signals associated with blood pressure measurements.

21. Blood pressure measurement apparatus according to any preceding claim, including means for storing the result of tie analysis of the received signal.

22. Blood pressure measurement apparatus according to any preceding clam when said analysing means comprises neural network means.

23. Blood pressure measurement apparatus according to any preceding claim, wherein said analysing means is adapted to extract features from the received signal giving morphological and rhythm information.

24. Blood pressure measurement apparatus according to any preceding claim wherein said blood pressure measuring means comprises oscillometric blood pressure measuring means.

25. Blood pure measurement apparatus according to any one of claim 1 to 23, wherein said blood pressure measuring means comprises auscultatory blood pressure measuring means.

26. Blood pressure measurement apparatus according to claim 25, wherein said blood pressure measuring means is adapted to make R wave Sued measures of blood pressure using the the received signal.

27. Blood pressure measurement apparatus according to any preceding claim, wherein said apparatus is portable and said blood pressure measuring means is an ambulatory blood pressure measuring means.

28. A method of measuring the blood pressure of a patient; the method Comprising:
receiving a signal from the patient indicative of the contractions of the heart of the patient;
analysing the received signal;
generating a blood pressure request signal;
determining whether to take a blood pressure measurement on the basis of the analysis of the received signal; and
taking a blood pressure measurement if said blood pressure measurement request signal has been generated in dependence upon the result of the determination.

29. A method according to claim 28, wherein the request signal is automatically periodically generated.

30. A method according to claim 28 or claim 29, wherein the request signal is manually generated.

31. A method according to any one of clams 28 to 30, wherein the analysis step identifies whether it is a suitable time for a blood pressure measurement to be taken, and a determination step determines that a blood pressure measurement is to be taken only if the analysing step has identified the time to be suitable.

32. A method according to claim 31, wherein the analysing step includes identifying cardiac conditions and/or cardiac rhythms and classify the cardiac conditions and/or cardiac rhythms as suitable or unstable for a blood pressure measurement.

33. A method according to claim 32, wherein the analysing step includes using a neural network for each of a plurality of cardiac classifications to classify the received signal based on its morphology, determining cardiac rhythm information from the received signal, and identifying cardiac conditions and/or cardiac rhythms using the cardiac classifications and the cardiac rhythm information.

34. A method according to any one of claims 28 to 33, wherein the analysing step includes continuing to analyse the received signal during a blood press measurement and a determination step includes determining whether a blood pressure measurement should continue or be aborted on the basis of the analysis of the received signal.

35. A method according to claim 34, wherein the analysing step includes identifying whether the functioning of the heart is stable for continuance of a blood pressure measurement and a determining step includes determining to continue to take blood pressure measurement only if the analysing step has identified the functioning of the heart to be suitable.

36. A method according to claim 35, wherein the analysing step includes identifying cardiac conditions and/or cardiac rhythms and classifying the cardiac conditions and/or cardiac rhythms as suitable or unsuitable for a blood pressure measurement.

37. A method according to any one of claims 34 to 36, wherein the determining step further includes determining whether to suspend a blood pressure measurement for a number of cardiac cycles on the basis of the analysis of the received signal.

38. A method according to any one of claims 28 to 37, including analysing the received signal to identify predetermined cardiac events for which a blood pressure measurement is desired, and taking a blood pressure measure when a said predetermined cardiac event is identified.

39. A method according to any one of claims 37 to 38, wherein the analysing step comprises continuously analysing the received signal.

40. A method according to any one of claim 28 to 37, wherein the analysing step comprises analysing the received signal only after said request signal is generated and whilst the blood pressure measurement is being taken.

41. A method according to any one of claims 28 to 40, including storing blood pressure measurements taken.

42. A method according to any one of claims 28 to 41, including storing the received signal.

43. A method according to claim 42, wherein only the received signal associated with a blood measurement is stared.

44. A method according to any one of claims 28 to 43, including storing a result of the analysis of the received signal.

45. A method according to any one of clam 28 to 44, wherein the analysing step uses neural network means.

46. A method according to any one of claims 28 to 45, wherein the analysing step includes extracting features from a received signal which give morphological and rhythm information.

47. A method according to any one of claims 28 to 46, ten The blood pressure measurement is to using oscillometric blood pressure means.

48. A method according to any one of claims 28 to 46, wherein the blood pressure is taken using auscultatory blood pressure measuring means.

49. A method according to claim 48, wherein the blood pressure is taken by making R wave gated measures of blood pressure using the received signal.

50. A method according to any one of claims 28 to 49, wherein the analysing step and the determining step are carried out in a portable apparatus and the blood pressure is taken using an ambulatory blood pressure measuring means.

51. A method according to any one of claims 28 to 50 wherein an electophysiological cardiac signal is received and analysed.

52. A method according to any one of claims 28 to 51 wherein the received signal is analysed to determine pulse rate and pulse intervals.

53. A method according to any one of claims 28 to 52 wherein the received signal is an ECG signal.
